(19) United States
(12) Patent Application Publication
(10) Pub. No.: US 2005/0272720 A1
(43) Pub. Date: Dec. 8, 2005

(54) Process for making olanzapine Form I

(76) Inventor: Rolf Keltjens, Nijmegen (NL)

Correspondence Address: MARK R. BUSCHER, P.O. BOX 161, CATHARPIN, VA, 20143, US

(21) Appl. No.: 11/050,851

(22) Filed: Jan. 27, 2005

Related U.S. Application Data
(60) Provisional application No. 60/539,120, filed on Jan. 27, 2004.
(60) Provisional application No. 60/562,225, filed on Apr. 15, 2004.

(51) Int. Cl. A61K 31/551; C07D 498/14

(52) U.S. Cl. 514/220; 540/557

(57) ABSTRACT

Heating a solid, preferably crystalline, olanzapine acetate can produce olanzapine Form I in high purity, free of other olanzapine forms and in good yields. The olanzapine acetate can also be used to purify raw or technical grade olanzapine and to serve as an intermediary to other forms of olanzapine base.

This application claims the benefit of priority from U.S. Provisional Application Ser. No. 60/539,120, filed Jan. 27, 2004, and from U.S. Provisional Application Ser. No. 60/562,225, filed Apr. 15, 2004; the entire contents of each application being incorporated herein by reference.

### BACKGROUND OF THE INVENTION

The present invention relates to a new method of making olanzapine in the crystalline Form I and to new salt or adduct useful therein. 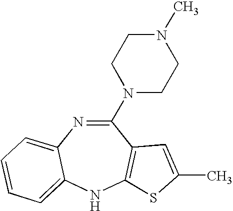
is a pharmaceutically useful compound. In medical treatments, it is useful as an antipsychotic agent, particularly for the treatment of schizophrenia; it acts as serotonin (5-HT2) and dopamine (D1/D2) receptor antagonist with anticholinergic activity. The marketed final forms include coated tablets and quick dissolvable tablets. The single tablet comprises from 2.5 to 20 mg of olanzapine

Olanzapine is an amine and may form acid addition salts. In commercially available final forms, however, the active substance is marketed as a free base. It is a white to yellow crystalline solid that is insoluble in water (solubility at pH 6.8=0.02 mg/ml).

Olanzapine and pharmaceutically acceptable salts have been suggested in EP 454436 and corresponding U.S. Pat. No. 5,229,382. The U.S. Pat. No. 5,229,382 does not refer to any specific polymorphic crystalline form of olanzapine.

U.S. Pat. No. 5,229,382 also describes a synthetic method for making crude olanzapine, wherein “des-methylpiperazine olanzapine” intermediate (as the HCl-salt usually) reacts with N-methylpiperazine in DMSO/toluene at enhanced temperature to make olanzapine. The reaction is illustrated by the following equation: 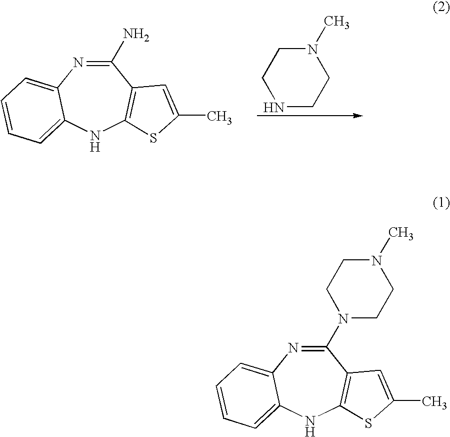
whereby the reaction mixture is treated with water and the product precipitates after cooling. The crude product has been recrystallized from acetonitrile.

Later, it became known that olanzapine base may exist in various crystalline modifications, including some hydrated/solvated forms, that are stable at ambient conditions (For example, see EP 733635/U.S. Pat. No. 5,736,541, WO 98-11893, and EP 831098).

The term “Form I olanzapine” was later designated in EP 733635 to the anhydrous olanzapine product that was stated to be obtainable according to the above process of U.S. Pat. No. 5,229,382.

EP 733635/U.S. Pat. No. 5,736,541 disclose Form II olanzapine which is characterized by a main X-ray powder diffraction peak of d-value 10.26 A. This form has been prepared by crystallizing “technical grade” olanzapine (a product from the above synthesis) from ethyl acetate. This form appears to be more stable than the Form I, but it is convertible to the said Form I. Similarly as Form I, the Form II is an anhydrate.

U.S. Pat. No. 6,348,458 (WO 01/47933) discloses other crystalline polymorphic forms of olanzapine, namely Form III, Form IV and Form V. More recently, WO 03/091260 discloses Form VI olanzapine. US appl. 2002-0086993 discloses a poymorphic form designated as form X.

As the system used for numbering of known olanzapine forms is sometimes confusing in the prior art disclosures (for instance, the EP 828494 calls as Olanzapine Form I a product that is identical with Olanzapine Form II of the above definition), the “Form I olanzapine ” is defined within this invention as the solid state form of anhydrous olanzapine base which is characterized by a main peak on the X-ray powder diffraction spectrum of d-value 9.9463 A. The full diffraction pattern of the Form I has been disclosed in EP 733635.

However, WO 02/18390 indicates that, upon repetition of the disclosed process, the product of U.S. Pat. No. '382 does not correspond to the Form I, but it is rather a Form II olanzapine (see above for definition) after the crystallization from acetonitrile, or a hydrated olanzapine prior said crystallization.

The Form I complying with the above definition was actually prepared in WO 02/18390 by recrystallization of olanzapine Form II or a hydrate of olanzapine from dichloromethane, followed by drying of the wet product at 60-70° C. In fact, the product of crystallization is a dichloromethane solvate of olanzapine, which liberates dichloromethane under the conditions of drying and yields the Form I.

Furthermore, Reutzel-Edens et al. ( Crystal Growth and Design, 2003, vol. 3, No. 6, 897-907) studied various solid state forms of anhydrous and hydrated forms of olanzapine. They have found out that while it is possible to prepare pure Olanzapine form II by a direct crystallization from various solvents (please note that such form is designed as “Form I” in that article), it is impossible to prepare Olanzapine Form I (designed as “Form II” in that article) in such a way. The Form I is obtainable only by a desolvation of various olanzapine solvates (methanol, dichloromethane and/or chloroform solvates) and such a product is admixed with various other forms of olanzapine. No conditions were identified that would yield pure form I.

WO 03/97650 purports to prepare essentially pure Olanzapine Form I by a desolvation of various olanzapine solvates. However, when looking on the published X-ray diffraction pattern of the product, the product is not the Olanzapine form I as above-defined.

Essentially pure Olanzapine Form I was prepared and characterised in WO 03/101997, employing a complicated purification and precipitation process.

WO 04/006933 attempts to prepare Olanzapine Form I by a desolvation of various solvates and mixed solvates.

The Form I olanzapine is still an important product so that an improvement in making it is desirable. In particular, it is desirable to provide essentially pure Olanzapine Form I, free from other polymorphic forms, by a simple and controllable process. It would also be advantageous to avoid the use of organic solvents, especially chlorinated solvents such as dichloromethane.

### SUMMARY OF THE INVENTION

The present invention relates to an olanzapine acetate and the uses thereof in forming olanzapine. In particular a first aspect of the invention relates to an olanzapine acetate, in any form including dissolved in a solvent, but preferably in solid, especially crystalline form. As used herein “olanzapine acetate” means the salt, adduct, or complex of olanzapine and acetic acid in substantially equimolar amounts. Thus a preferred substance of the invention is a crystalline compound comprising substantially equimolar amounts of olanzapine and acetic acid, and/or the respective ions thereof.

Another aspect of the invention relates to a process of making olanzapine acetate in solid form, which comprises contacting a source of olanzapine with acetic acid in a solvent, and precipitating the olanzapine acetate from the solvent. Preferably the solvent is acetone. The “source of olanzapine” includes olanzapine base, in any solid state form or as a solvate, or a reaction mixture comprising olanzapine base as the product of a chemical reaction.

A further aspect of the invention relates to a process for making olanzapine Form I, which comprises heating a solid state olanzapine acetate at a temperature within the range of 50-120° C. for a sufficient time to form olanzapine Form I. The heating is preferably under diminished pressure or vacuum or under other means that aid the removal of the liberated acetic acid from the remaining olanzapine such as an inert gas purge. The product of the heating step (thermal decomposition) is olanzapine form I. The process typically provides olanzapine Form I substantially free form other forms of olanzapine, including hydrates and solvates.

An additional aspect of the invention relates to a process of purifying of olanzapine, which comprises converting olanzapine into olanzapine acetate, isolating said olanzapine acetate and converting the olanzapine acetate to olanzapine. The conversion can be by heating to form olanzapine Form I or by reaction with base to form olanzapine with optional further precipitation to a desired solid form.

### BRIEF DESCRIPTION OF THE DRAWINGS

FIG. 1 is an x-ray powder diffraction pattern (XRPD) corresponding to the olanzapine acetate of example 3.

FIG. 2 is an x-ray powder diffraction pattern (XRPD) corresponding to the olanzapine Form I produced in example 4.

### DETAILED DESCRIPTION OF THE INVENTION

The present invention is based on the discovery that olanzapine acetate can be converted to olanzapine Form I by heating. Surprisingly, not only is the thermal degradation into olanzapine Form I possible, but generally a substantially pure Form I is obtained, free of the other olanzapine forms.

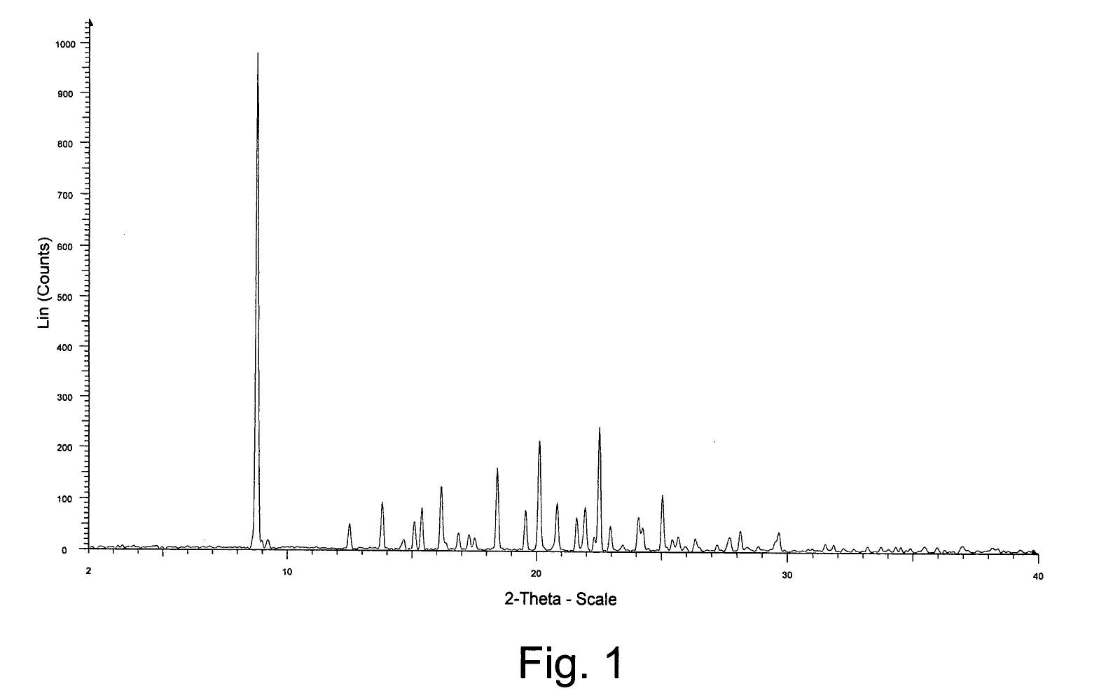

The olanzapine acetate in solid state is a particulate material, with no or minimal tendency of reversible absorption of water from the environment, and with good handling properties. It is sufficiently stable under normal conditions of temperature and moisture. In particular, the solid material may exhibit substantially white to yellow color. Typically, it exhibits an X-ray powder diffractogram as shown in FIG. 1.

Olanzapine acetate of the present invention is presumed to be a salt. The acetic acid, being a weak acid, reacts with essentially only one of the basic nitrogens in the olanzapine ring structure to form a molar ratio of olanzapine (or ion thereof) to acetic acid (or ion thereof) of about 1:1 ±0.1. Its chemical formula can be represented as: 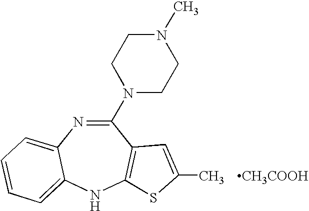
However, unlike a typical salt, the acetic acid is removable by heating to form the olanzapine base in Form I. Whether the acetate salt converts to a solvate and then undergoes desolvation, or, is simply not a true salt but rather is an adduct, complex, solvate, etc. is not entirely clear. Accordingly, the “olanzapine acetate” as used in the present invention embraces traditional salts of olanzapine and acetic acid as well as adducts, complexes, solvates, etc.

The ability of the acetate to thermally convert to olanzapine base is apparently unique. For example, formic acid, another weak acid and smaller in size than acetic acid, also forms a salt with olanzapine, but no thermal conversion to olanzapine base is observed upon heating such a formate.

Regardless of the true nature of the product, the solid state olanzapine acetate of the present invention is a compound that can yield olanzapine base after thermal treatment. Under typical conditions, solid state, especially crystalline olanzapine acetate is placed in the heated vessel, without employing any solvent or catalyst, and is heated to a temperature between 50° C. and 120° C., preferably between about 70-100° C., for a sufficient time to substantially remove all the acetic acid from the vessel. The liberated acetic acid is advantageously removed from the vessel with the aid of diminished pressure, especially vacuum or by a stream of inert gas, the first being preferred.

If necessary (particularly from the safety and ecology aspects), the liberated gaseous acetic acid removed from the vessel may be trapped into a solvent, adsorbed on a column with a suitable adsorbent, condensed by cooling in a cooling trap, or neutralized in a washer filled by a solution of appropriate base.

The product of the heating (and presumed thermal decomposition) of olanzapine acetate is Olanzapine Form I. Such form, as known from documents of the prior art, was made so far only by drying the dichloromethane solvate of olanzapine. As no solvent has been employed in the process of the present invention and the decomposition temperature is lower than the transition temperature at which the Form I is converted into Form II (such transition temperature is about 125° C. according to the article, crystal growth & design, 2003, 3, 897-907), the produced olanzapine Form I is typically substantially free from other solid state forms, hydrates and /or solvates. In particular, the content of the Form I is higher than 99%. The yield of olanzapine Form I after thermal conversion of olanzapine acetate is substantially quantitative.

A common method of making the olanzapine acetate in solid state is contacting a source of olanzapine with acetic acid in a solvent, and precipitating the olanzapine acetate from said solvent. The aqueous solubility of olanzapine acetate limits the number of useful solvent systems. Preferably, such solvent system should not comprise water but this does not preclude using mixtures of nonaqueous solvents with water in the process. Suitable solvents comprise C1-C6 aliphatic ketones, C1-C6 ethers (incl. cyclic ethers) or C1-C6 esters. The most useful solvent in making the olanzapine acetate is acetone. Olanzapine base and sources thereof are generally sufficiently soluble therein as well as acetic acid, while olanzapine acetate is only slightly soluble in acetone. Thus acetone is a good solvent for affording contacting of the olanzapine base material and acetic acid as well as for facilitating the subsequent precipitation or crystallization of the olanzapine acetate. Other useful solvents are ethylacetate and tetrahydrofuran.

The solubility of starting materials in the solvent may be enhanced by raising the temperature of the reaction mixture, so that the salt forming reaction may proceed at a temperature from ambient to a boiling point of the solvent. Advantageously, the so formed solution of the salt may be treated with a suitable adsorption material such as activated charcoal, to remove contaminants.

The solubility of the salt product in the solvent system may be, accordingly, decreased by cooling the reaction mixture. In general, precipitation from the solvent is preferred to be spontaneous at the temperature of salt formation or to proceed after cooling. In addition, the precipitation may be forced by reducing the volume of the solvent, seeding, adding a contrasolvent or combination of these techniques.

Any grade or form of olanzapine is useful in the above process for making olanzapine acetate. For instance, the “technical grade” olanzapine (this material comprises also solvates of olanzapine with various solvents), Olanzapine Form II, Olanzapine hydrate etc.

Further more, the olanzapine acetate may be also prepared by a direct synthesis of the olanzapine moiety. For instance, the des-methyl olanzapine (3) may be methylated by formic acid/formaldehyde (Eschweiler-Clarke reaction) under the equation 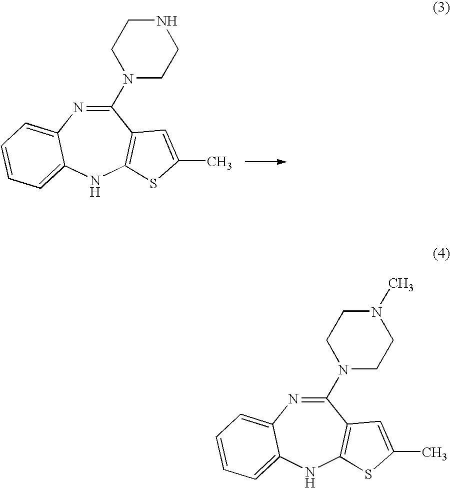
and the reaction mixture comprising olanzapine is treated with the acetic acid, under conditions that olanzapine salt, in his case olanzapine acetate, precipitates from the mixture. Alternately, the “des-piperazine olanzapine (2)” may be treated with N-methylpiperazine to yield olanzapine within a reaction mixture, as shown above, and such reaction mixture, after necessary elaboration, is treated with acetic acid under conditions of precipitation of the olanzapine acetate from it.

Furthermore, olanzapine acetate represents a suitable tool for purifying olanzapine, particularly the technical grade olanzapine. The process generally comprises converting olanzapine into olanzapine acetate, isolating the olanzapine acetate and converting the olanzapine acetate to olanzapine. The isolation of olanzapine acetate is normally into a solid state, especially a crystalline state, but such is not necessarily required. A preferred process comprises the step of transforming technical grade olanzapine base into olanzapine acetate in a non-aqueous solvent. When combined with the further step of heating the formed and isolated olanzapine acetate back to the olanzapine base, the technical grade olanzapine is efficiently purified from contaminants. This process may remove contaminants which are hardly removable by crystallization and generally avoids enhanced temperatures (a source of decomposition) that are required in routine crystallization procedures. The conversion of the olanzapine acetate back to olanzapine base can also be arranged by reaction with a base in solvent. The free base of olanzapine may precipitate or be further processed. Dependent on the conversion conditions, various forms of olanzapine in improved purity may be obtained. For instance, as shown above, thermal decomposition of olanzapine acetate will yield olanzapine form I. Reaction of the olanzapine acetate with the base in an aqueous medium generally yields olanzapine hydrate. Reaction of olanzapine acetate with a base in an alcoholic solvent generally yields olanzapine alcoholates, i.e., methanolate or isopropanolate.

The invention will be further described by the following non-limiting examples.

### EXAMPLE 1

### Olanzapine acetate

To a solution of 5.0 g of olanzapine base in 150 ml acetone was added slowly 1.06 g of acetic acid at room temperature and the mixture was stirred overnight at 4° C. The crystals were isolated by filtration, washed with 20 ml acetone and 20 ml of ether and dried overnight at 40° C. in vacuo.

Yield: 3.32 g (56%)

### EXAMPLE 2

### Olanzapine acetate

To 10.0 g of olanzapine free base dissolved in 200 ml of acetone was added slowly 3.85 g of acetic acid at room temperature. The resulting mixture was stirred overnight at 4° C., and the crystals were isolated by filtration, washes with diethyl ether and dried overnight at 40° C. in vacuum.

### EXAMPLE 3

### Olanzapine acetate

To a clear solution of 1.0 g of olanzapine free base in 40 ml ethyl acetate was added at room temperature 0.38 g acetic acid. The resulting clear mixture was seeded and subsequently stirred overnight at 4° C. The crystals were isolated by filtration and dried overnight at 40° C. in vacuo.

Isolated yield: 0.94 gram (79%)

The XRPD is shown in FIG. 1.

### EXAMPLE 4

### Olanzapine Form I

In a 10 ml flask, 0.5 g of olanzapine acetate was stored at 65-70° C. in vacuo to remove the acetic acid. After approximately 18 hours, the acetate was removed from the sample as was shown using 1H-NMR spectroscopy.

Isolated yield: 0.420 gram (100%)

¹H-NMR: no acetic acid present.

XRPD, IR, DSC: OPN free base Form I

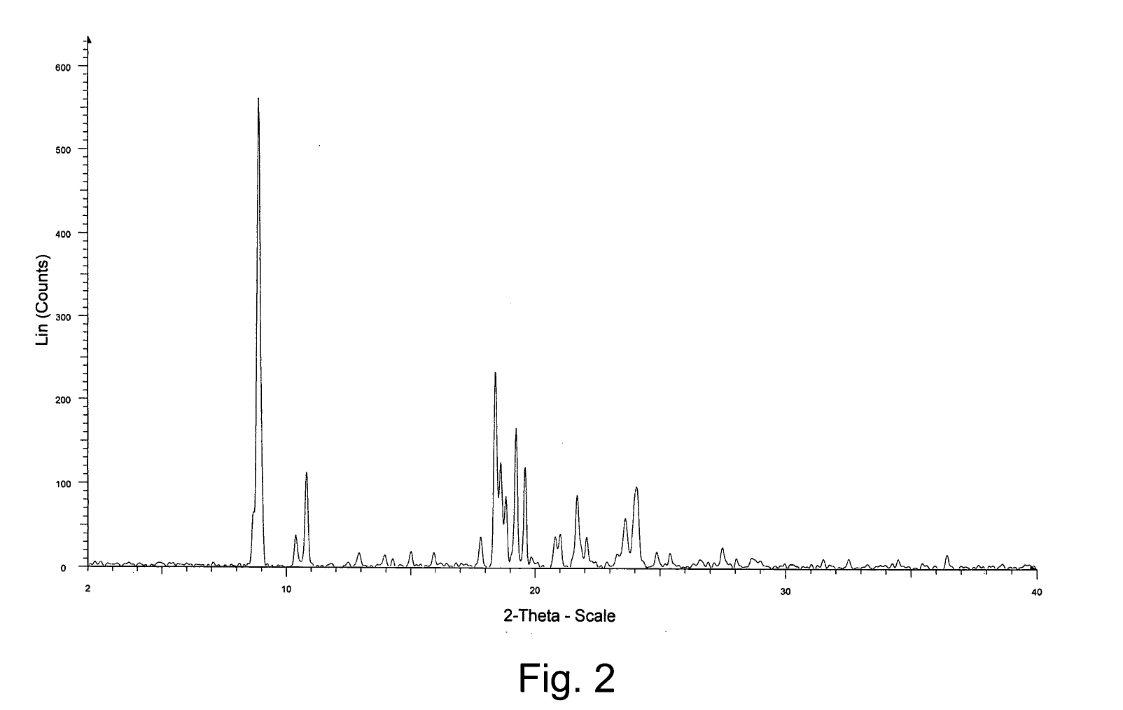

The XRPD is shown in FIG. 2.

### EXAMPLE 5

### N-Methylation of N-desmethyl olanzapine and isolation of olanzapine as the acetate salt

Reaction scheme:

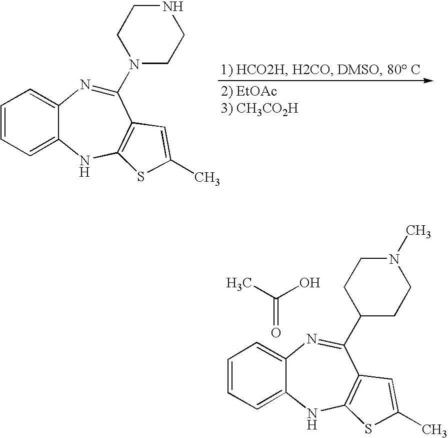

In a 250 ml flask, equipped with a magnetic stirrer 3.0 g of N-desmethyl olanzapine was dissolved in 30 ml of DMSO. Then, 0.852 g of 37% formaldehyde and 0.492 g of formic acid were added and the resulting mixture was heated at 80° C. for 2 hours. After cooling to room temperature, 120 ml of ethyl acetate and 120 ml of water were added. The organic layer was washed twice with 100 ml water and once with 100 ml saturated aqueous NaCl, dried over Na₂SO₄, and concentrated at reduced pressure to a volume of about 25 ml. Then, 1.0 ml acetic acid was added, and the resulting mixture was stirred for 3 days at 4° C. The product (light yellow) was isolated by filtration, and washed with 5 ml of diethyl ether, and dried subsequently at ambient conditions. Isolated yield: 1.19 g (58%) (=. batch.01).

Then, drying of 0.40 g of batch.01 in vacuum at 70° C. for 2 days yielded olanzapine free base quantitatively. Data: ¹H-NMR and XRPD confirm Olanzapine Form I.

### EXAMPLE 6

### Synthesis of olanzapine (final step) and isolation of olanzapine as the acetate salt

Reaction scheme:

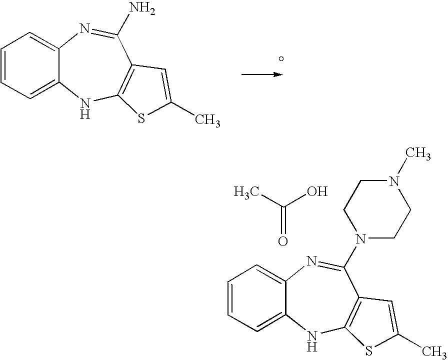

In a 250 ml flask, equipped with a magnetic stirrer, 4.3 g of N-desmethylpiperazine-olanzapine was refluxed in a mixture of 15 ml N-methylpiperazine, 20 ml DMSO, and 20 ml toluene under a nitrogen atmosphere for 20 hours. The mixture was cooled and 50 ml of water was added. The aqueous layer was extracted three times with 150 ml of ethyl acetate and the combined organic layers were washed 3 times with 100 ml water and once with 100 ml aqueous saturated sodium chloride. After drying over Na₂SO₄, the organic layer was concentrated to about 25 ml and 1.0 ml acetic acid was added. The resulting dark brown mixture was stirred at 4° C. for 2 hours. The solid material (light yellow) was isolated by filtration, and washed with 5-10 ml of diethyl ether, and dried overnight at 40° C. in vacuum. Isolated yield: 3.49 g (58%) (=batch.01).

### EXAMPLE 7

### Preparation of olanzapine base and olanzapine acetate from N-desmethylpiperazine olanzapine; comparison of purity

Reaction scheme:

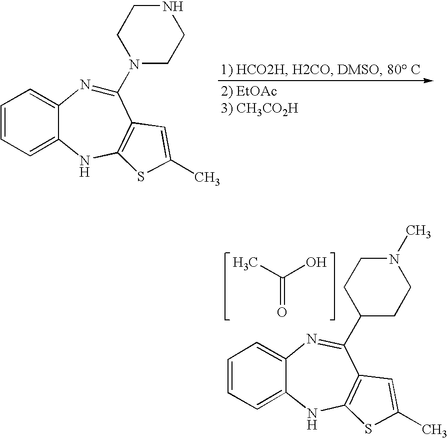

In a 250 ml flask, equipped with a magnetic stirrer, crude 4.3 g of N-desmethylpiperazine-olanzapine was refluxed in a mixture of 30 ml N-methylpiperazine, 40 ml DMSO, and 40 ml toluene under a nitrogen atmosphere for 20 hours. The mixture was cooled to about 50° C., and divided into two equal portions (each about 60 ml).

Portion 1 ( elaborated as in EP 454.436):

To portion 1, 20 ml water was added, and the product was allowed to crystallize at 5° C. overnight. The product was filtered and dried at 40° C. overnight.

Isolated yield: 4.70 g (=batch.01)

¹H-NMR: olanzapine base, 0.5 mol. equivalent of DMSO present

Portion 2:

To portion 2, 50 ml water were added and the aqueous layer was extracted three times with 150 ml ethyl acetate. The combined organic layers were washed 3 times with 100 ml water and once with 100 ml aqueous saturated sodium chloride. After drying (Na₂SO₄), the organic layer was concentrated to a volume of about 25 ml and 1.2 ml of acetic acid was added. The resulting clear brown (dark) mixture was stirred at 4° C. for 4 hours. The solid material was isolated by filtration, and washed with 5 ml ethyl acetate and 10 ml diethyl ether, and dried overnight at ambient conditions.

Isolated yield: 3.35 g (=batch.02).

¹H-NMR: olanzapine acetate

Remarks:

Batch.01—olanzapine base
very bad filterable (slurry)
(dark) brown color
0.5 mol. equivalent of DMSO present (¹H-NMR), possible solvate
purity (HPLC): 98.25%

Batch.02—olanzapine acetate
easy filterable (nice crystals)
light yellow solid
no solvent present (¹H-NMR), present as acetate
purity (HPLC): 99.04%

Each of the patents, articles, and publications mentioned above is incorporated herein by reference in its entirety. The invention having been thus described, it will be obvious to the worker skilled in the art that the same may be varied in many ways without departing from the spirit of the invention and all such modifications are included within the scope of the present invention as set forth in the following claims.

1. An olanzapine acetate.

2. The olanzapine acetate according to claim I in solid state.

3. The olanzapine acetate according to claim I in a solid crystalline form.

4. A crystalline compound comprising substantially equimolar amounts of olanzapine and acetic acid, and/or the respective ions thereof.

5. A process of making the compound of claim I in solid form, which comprises contacting a source of olanzapine with acetic acid in a solvent, and precipitating the olanzapine acetate from said solvent.

6. The process according to claim 5, wherein said solvent is acetone.

7. The process according to claim 5 wherein the source of olanzapine is a solid state olanzapine.

8. The process according to claim 5, wherein the source of olanzapine is a reaction mixture comprising olanzapine.

9. A process of making olanzapine Form I, which comprises heating a solid state olanzapine acetate at a temperature within the range of 50-120° C. for a sufficient time to form olanzapine Form I.

10. The process according to claim 9, wherein the heating is performed under diminished pressure.

11. The process according to claim 9, wherein the heating is performed under an inert gas purge.

12. A process of purifying of olanzapine, which comprises converting olanzapine into olanzapine acetate, isolating said olanzapine acetate and converting the olanzapine acetate to olanzapine.

13. The process according to claim 12, wherein said converting of said olanzapine acetate is carried out by thermal degradation and said olanzapine formed is olanzapine Form I.

14. The process according to claim 12, wherein said converting of said olanzapine acetate is carried out by reaction with a base to form said olanzapine.
